(19) United States
(12) Patent Application Publication
(10) Pub. No.: US 2026/0073502 A1
(43) Pub. Date: Mar. 12, 2026

(54) CATEGORIZING LOGS BASED ON GROWTH CHARACTERISTICS, AND ASSOCIATED SYSTEMS, DEVICES, AND METHODS

(71) Applicant: Weyerhaeuser NR Company, Seattle, WA (US)

(72) Inventor: David Irving, Boise, ID (US)

(21) Appl. No.: 19/325,087

(22) Filed: Sep. 10, 2025

Related U.S. Application Data
(60) Provisional application No. 63/693,087, filed on Sep. 10, 2024.

(51) Int. Cl. G06T 7/00; G01N 33/00

(52) U.S. Cl. CPC G06T 7/0004; G01N 33/0098; G06T 2207/30161

(57) ABSTRACT

Methods and systems of categorizing logs based on growth characteristics of the logs are disclosed. An exemplary method includes obtaining an image of an end surface of a log that includes growth rings, identifying one of more growth characteristics of the log based on the growth rings, and providing instructions to categorize the log based on the identified growth characteristics. The growth characteristics can include log age, diameter, rings per inch, and pith eccentricity. In some embodiments, images of both end surfaces of the log are obtained to identify other characteristics, such as an end-to-end diameter, which is used to provide further instructions to categorize the log.

### CROSS-REFERENCE TO RELATED APPLICATION

This application claims the benefit of U.S. Provisional Patent Application No. 63/693,087, filed Sep. 10, 2024, and titled “METHODS AND SYSTEMS OF CATEGORIZING LOGS BASED ON GROWTH CHARACTERISTICS,” which is incorporated herein by reference in its entirety.

### TECHNICAL FIELD

The present disclosure is generally related to categorizing logs based on growth characteristics, and associated systems, devices, and methods.

### BACKGROUND

Wood products are manufactured by processing logs through a mill. One aspect of processing logs is merchandising the logs, which involves sorting them into various categories based on size, quality, and intended end-use. Merchandising seeks to enhance the value extracted from each log by directing it to the most suitable processing path, whether it be for finished lumber, veneer, pulp, or manufactured wood products (e.g., particle board, plywood, etc.). Ensuring that each log is allocated along the correct processing path reduces waste and increases profitability.

In some applications, the commercial value of a given log is tied to its stiffness and/or strength. For example, structural timber used in construction must meet specific strength and stiffness criteria to ensure the safety and stability of buildings. The ability to estimate these properties on raw logs may ensure they are directed towards the best suited products, otherwise inferior-strength logs can be mistakenly processed for high value construction purposes while superior-strength logs may end up being processed for lower value wood products. Additionally, traditional methods of determining the stiffness and/or strength of logs may rely on manual inspection, which is time-consuming and prone to human error and inconsistency. Furthermore, determining stiffness and strength can be difficult because the logs are often stored in large piles outside of a mill where they are dirty and the end cuts are rough. Thus, there is a need to improve the identification and sorting of logs to the most appropriate processing path.

### BRIEF DESCRIPTION OF THE DRAWINGS

FIG. 1A is a partially-schematic perspective view of a system for identifying log characteristics, in accordance with some embodiments of the present technology.

FIG. 1B is a partially-schematic view of the system of FIG. 1A, in accordance with some embodiments of the present technology.

FIG. 2 is an illustration of an obtained image of an end surface of a log, in accordance with some embodiments of the present technology.

FIG. 3 is an illustration of an obtained image of an end surface of a log, in accordance with some embodiments of the present technology.

FIG. 4 is an illustration of an obtained image of the latewood and earlywood of part of an end surface of a log, in accordance with some embodiments of the present technology.

FIG. 5 is a table with log characteristics used for categorizations, in accordance with some embodiments of the present technology.

FIGS. 6-9 are methods of categorizing logs, in accordance with some embodiments of the present technology.

FIG. 10 is a system diagram illustrating an example of a computing environment in which the disclosed system operates, in accordance with some embodiments of the present technology.

FIG. 11 is a block diagram illustrating an example machine learning (ML) system, in accordance with some embodiments of the present technology.

FIG. 12 is a block diagram illustrating an example computer system, in accordance with some embodiments of the present technology.

The drawings have not necessarily been drawn to scale. Similarly, some components and/or operations may be separated into different blocks or combined into a single block for the purposes of discussion of some of the embodiments of the disclosed system. Moreover, while the technology is amenable to various modifications and alternative forms, specific embodiments have been shown by way of example in the drawings and are described in detail below. The intention, however, is not to limit the technology to the particular embodiments described. On the contrary, the technology is intended to cover all modifications, equivalents and alternatives falling within the scope of the technology as defined by the appended claims.

### DETAILED DESCRIPTION

The present technology is directed to systems, devices, and methods for categorizing logs of ring-porous tree species (i.e., species where latewood bands are distinct from earlywood bands) based on growth characteristics of the log. In the wood products industry, high strength and high stiffness logs are commercially valuable for certain uses. To determine the overall quality of logs (e.g., based on strength and stiffness), commercial log merchandisers often rely on externally measured characteristics such as sweep (the curvature or bend in the log), knot whorls and/or bulges, small end diameter (SED), large end diameter (LED), and log length. Growth characteristics, such as growth rings and pith, are not typically used to characterize logs for merchandising purposes because existing practices for identifying such features are cumbersome, prone to error, and time consuming. Furthermore, conventional technology is often inaccurate at identifying growth characteristics of certain logs (e.g., logs that were recently harvested and/or have a high moisture content, or logs for which a relatively long time has elapsed since harvesting and/or have a very low moisture content), and can be exceedingly expensive to deploy.

The present technology addresses these and other related issues by providing systems, devices, and methods of categorizing logs into particular categories, such as high quality and/or strength, medium quality and/or strength, and low quality and/or strength. The categorization can be at least partly automated and/or make use of imaging technology to identify growth characteristics which, in some embodiments, are used to determine and/or predict mechanical properties of the log. Exemplary systems, devices, and methods obtain an image (e.g., via an imaging device) of an end surface (e.g., cross-cut end) of a log. In some embodiments, the image of the end surface includes the log's pith and growth rings. The systems, devices, and/or methods can automatically identify one or more growth characteristics (e.g., the growth rings and/or location of the pith) of the log based on the obtained image, using a computer (e.g., via image processing software), and in some embodiments categorize the log (e.g., as high strength/quality, medium strength/quality, low strength/quality), based on the identified growth characteristics.

Using imaging techniques and image processing technology to automatically identify and determine the growth rings, location of the pith, and other growth characteristics of the log (e.g., diameter, geometric center of end surfaces (C), rings per inch (RPI), eccentricity (E), latewood-to-earlywood ratio (LW/EW), percent latewood (LW %) and/or ovality) enables the present technology to efficiently and accurately identify high strength and high stiffness logs for commercial use, as well as excluding low strength and low stiffness logs for commercial use. The use of imaging technology, such as specialized lighting or hyperspectral cameras, simplifies the process of capturing consistent, clear images of log ends even when the log ends are cut at irregular and/or inconsistent angles, such as when there is considerable shadowing error (e.g., from uneven lighting conditions found in ambient or industrial environments) and/or when log surfaces include significant surface contamination that obscures the image.

Another example benefit of the present technology is that the identification of growth characteristics and/or accuracy of determination of stiffness and strength is not dependent on certain factors, such as the time elapsed since the timber is harvested with corresponding changes in the moisture content of the logs. Conventional technologies (e.g., those which do not directly measure growth rings on the ends of logs) experience diminished accuracy in identifying growth ring characteristics when logs exhibit moisture levels above the fiber saturation point. While the fiber saturation point itself can vary between individual trees and species, it is generally recognized that moisture contents exceeding approximately 25-30% (based on a comparison of the weight of the log (i.e., the “wet weight” of the log) with the oven-dry weight of the log) begin to adversely affect the reliability of conventional systems in measuring growth rings and pith location. In contrast, the present technology is agnostic with respect to moisture content of the log, enabling precise identification of growth characteristics and, by extension, accurate determination of mechanical properties in logs with high moisture content (e.g., greater than 30%, 40%, 50%, 100% etc.), as well as in logs with low moisture content (e.g., at or below the fiber saturation point). For example, the present technology can accurately determine the mechanical properties of logs with moisture content at least approximately 0%, 10%, 15%, 20%, 25%, 30%, 35%, 40%, or 50%, or within a range of approximately 0%-50% or any increment therebetween (e.g., 45%).

This independence from both moisture content and time elapsed since harvest has additional advantages. For example, acoustic velocity measurement can provide a relatively good indication of log stiffness and strength properties but is only a reliable predictor when logs are freshly harvested. Within three to five days, the moisture profile in the logs will begin to change, rendering acoustic techniques useless for the purpose of merchandising logs into defined stiffness and strength categories. In contrast, the present technology maintains its precision regardless of whether the logs are freshly harvested or have been stored for extended periods, and regardless of whether the moisture content is low or high. For example, the present technology can accurately determine the mechanical properties of logs with a time elapsed since harvest of approximately 1 day, 3 days, 7 days, 10 days, 15 days, 30 days, or greater (e.g., approximately 6 months, 1 year, etc.).

For purposes of this application, the terms “growth characteristics” and “end surface characteristics” can refer to features of a log that are observable (e.g., via the imaging devices disclosed herein) from an end surface (e.g., either of the ends where the log was cut during harvesting) and/or other subsequent cuts on the log as it is further processed. The term “primary” growth characteristics can refer to observable characteristics associated with determining the number of growth rings (for example, ring count, rings per inch, pith location, bark layer location, end surface diameter, etc.). The term “secondary” growth characteristics can refer to characteristics not associated with determining the number of growth rings, but that are none-the-less observable from the end surface of the log. For example, secondary growth characteristics can include pith symmetry, pith eccentricity, LW/EW ratio, percent LW, percent EW, ovality etc. The term “supplemental” characteristics can refer to log characteristics that are (i) internal to the log but not visible to the naked eye without penetration of the outer surface (e.g., due to being obscured by the bark or other outer layers of the log), and/or (ii) are associated with the external surface and shape of the overall log, (e.g., at least a portion of the curved external surface/outer bark layer of the log). For example, supplemental characteristics can include log sweep, log length, size of knot whorls and/or surface bulges, quantity of knot whorls and/or surface bulges, bow, taper, crook, decay, cracks, surface damage, etc.

### Methods and Systems of Categorizing Logs

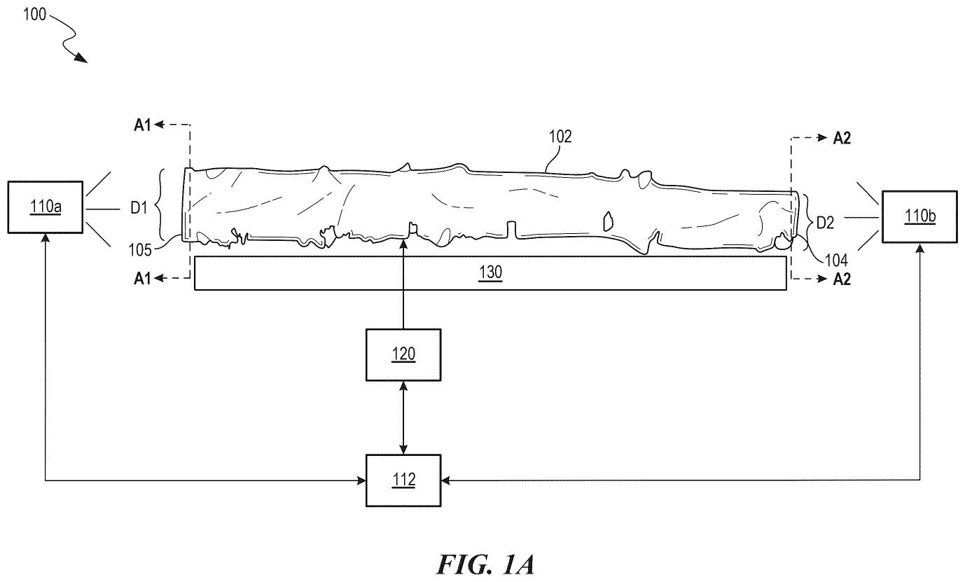

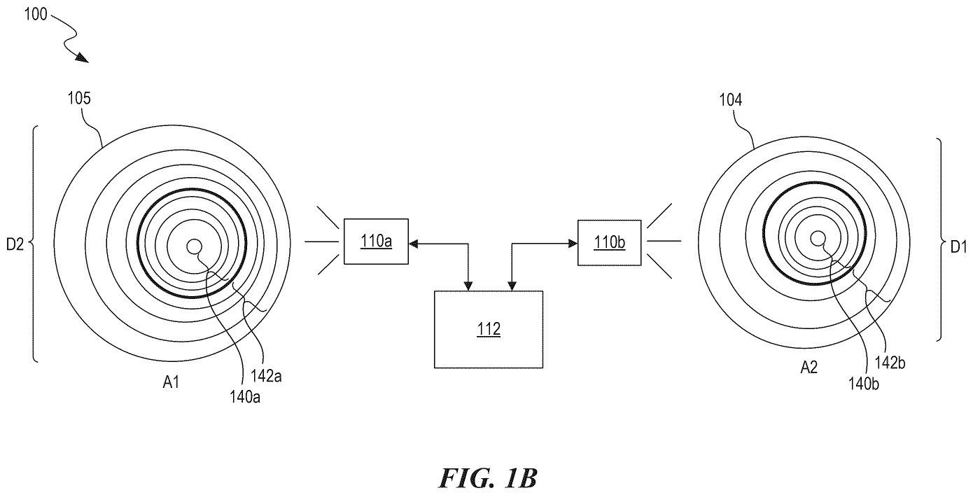

FIG. 1A is a partially-schematic perspective view of a system 100 for identifying characteristics of a log 102 in accordance with some embodiments of the present technology. In some embodiments, the system 100 comprises a first imaging device 110a and a second imaging device 110b (collectively referred to as imaging device 110), each configured to obtain respective end surface characteristics of the log 102, and a computer device 112 operably coupled to the imaging device 110. The first imaging device 110a is configured (e.g., positioned) to obtain or capture one or more images of a first end surface 104 of the log 102 and the second imagine device 110b is configured to obtain or capture one or more images of a second (opposing) end surface 105 of the log 102. As shown in FIG. 1A, the system 100 can further comprise one or more measurement devices 120 operably coupled to the computer device 112 and configured to obtain supplemental characteristics (discussed further herein) of the log 102. In some embodiments, the system 100 includes one or more platforms 130 configured to physically support, transport, rotate, and/or move the log 102 or a plurality of logs as needed to obtain characteristics thereof.

In some embodiments, the system 100 obtains images of only one of the end surfaces 105, 104 via the corresponding imaging device 110. The image(s) can be taken along a longitudinal axis of the log 102 that is approximately perpendicular to the corresponding end surface 104, 105 of the log 102. For example, the first imaging device 110a can capture an image of the first end surface 105 that is along axis A1 approximately perpendicular to the first end surface 105. A second imaging device 110b can capture an image of the second end surface 104 that is along axis A2 approximately perpendicular to the second end surface 104. In some embodiments, the first end surface 105 has a first diameter D1 and the second end surface 104 has a second diameter D2 less than D1. In such embodiments, D1 can be referred to as the LED and D2 can be referred to as the SED. The first diameter D1 and/or LED (or diameters generally) can be at least 6 inches, 8 inches, 10 inches, 12 inches, 14 inches, 16 inches, 18 inches, or 20 inches, and the second diameter D2 and/or SED (or diameters generally) can be at least 4 includes, 6 inches, 8 inches, 10 inches, 12 inches, 14 inches, or 16 inches.

In some embodiments, the system 100 obtains an image that depicts only a portion of the first and/or second end surfaces 105, 104 (also referred to as a “partial image”). For example, the image can depict no more than approximately 10%, 20, 30%, 40%, 50%, 60%, 70%, 80%, 90%, etc., or within a range of 10-90% or any increment therebetween (e.g., 50%) of the surface area of the corresponding first and/or second end surfaces 105, 104. For example, imaging device 110a can obtain a first image depicting the growth rings of only an inner portion 140a (as shown in FIG. 1B), which can be used to determine an inner portion 140a RPI, and a second image depicting the growth rings of only an outer portion 142a (as shown in FIG. 1B), which can be used to determine an outer portion 142a RPI. The inner portion 140a RPI and/or outer portion 142a RPI can be analyzed to determine, for example, strength and stiffness of the log 102, and/or to categorize the log.

In some embodiments, the imaging device 110 obtains a plurality of images of at least one of the first and/or second end surfaces 105, 104, which are then analyzed to determine the growth characteristics of the log 102 (e.g., via computer device 112 discussed further herein). In some embodiments, at least one of the images depicts only a portion of the first and/or second end surfaces 105, 104, as described herein. For example, at least one of the images of the plurality of images can depict no more than approximately 10%, 20%, 30%, 40%, 50%, 60%, 70%, 80%, 90%, etc., or within a range of 10-90% or any increment therebetween (e.g., 25%) of the surface area of the corresponding first and/or second end surface 105, 104. In some embodiments, at least one of the images is angled with respect to the axis A1 and/or A2. For example, at least one of the plurality of images can be at least approximately 10 degrees, 20 degrees, 30 degrees, 40 degrees, 50 degrees, 60 degrees, 70 degrees, 80 degrees, etc., or within a range of approximately 10-80 degrees, or any increment therebetween (e.g., 45 degrees) with respect to the corresponding axis A1, A2. In such embodiments, the partial images and/or angled images can be analyzed (e.g., via computer processing software and/or algorithms) to construct a virtual rendering or model of one or more of the first and/or second end surfaces 105, 104, which can be subsequently processed to identify and/or determine, for example, one or more growth characteristics of the log 102.

In some embodiments, the imaging device 110 includes cameras that capture image data in the red-green-blue (RGB) spectrum (e.g., visible light camera devices). The imaging device 110 can include hyperspectral imaging devices (e.g., imaging devices that capture a broad range of spectrum data for each pixel in an image, including visible light, infrared, and ultraviolet wavelengths), handheld cameras (e.g., phone cameras), and/or other imaging devices (e.g., laser arrays, photo arrays, light field imaging devices, and/or surface scanning devices). In some embodiments, one or more of the imaging devices 110 include imaging devices and techniques configured to produce image data that, when processed via processing software and/or algorithms (discussed further herein), generate a three-dimensional (3-D) image of one or more of the first and/or second end surfaces 105, 104. For example, the imaging devices 110a, 110b can include x-ray detectors configured to detect x-rays, which are used to generate a computed tomography (CT) scan of the end surfaces 105, 104.

The images of the end surfaces 105, 104 of the log 102 can be obtained from a computer-based storage medium, such as a computer hard drive or the like, and/or obtained from an online storage medium, such as via an online database, cloud network, or the like. In such embodiments, the computer device 112 (discussed further herein) can identify, for example, one or more growth characteristics of the log 102 based on one or more images obtained via the imaging devices 110a, 110b, one or more images obtained via the computer-based storage medium, or both.

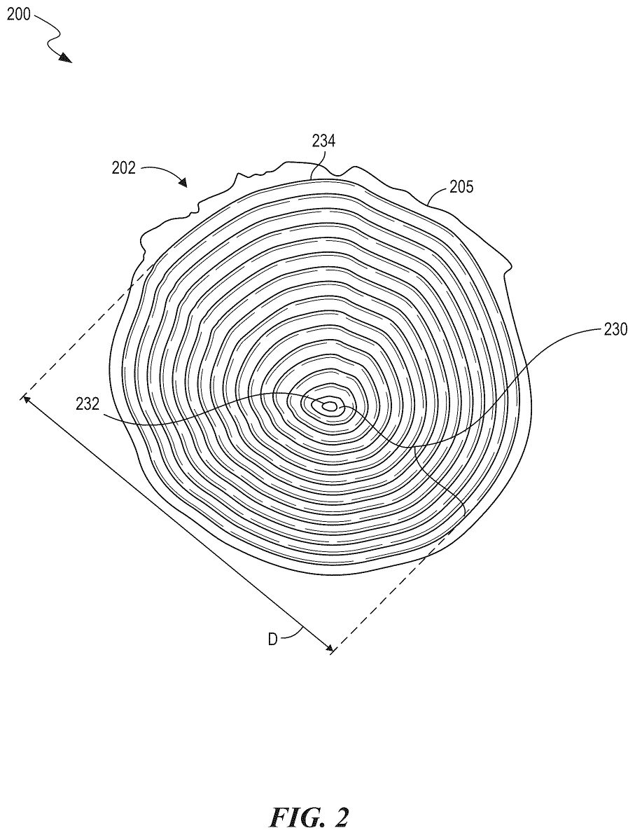

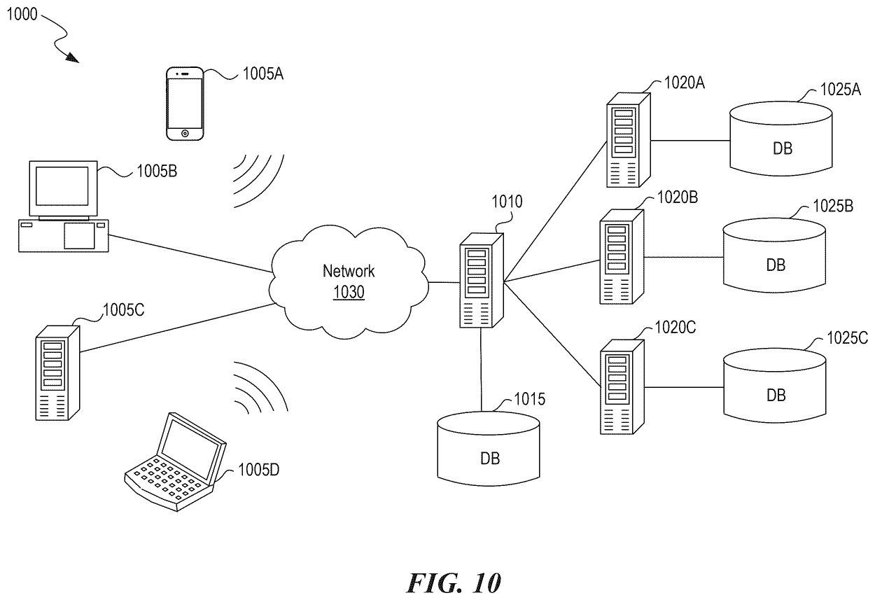

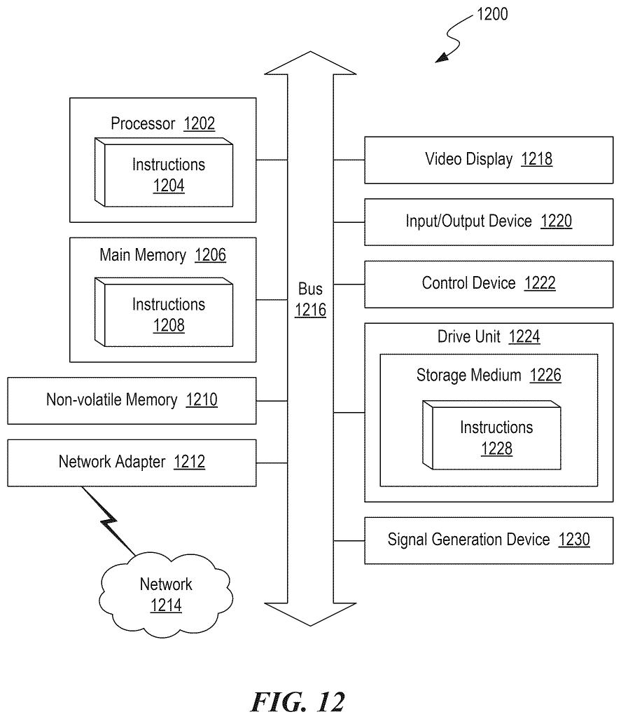

In some embodiments the images obtained are provided to the computer device 112 (e.g., any of the computer devices 1305A-D discussed further with reference to FIG. 10, and/or computer system 1200 discussed further with reference to FIG. 12). In some embodiments, the computer device 112 is communicatively coupled to the one or more imaging devices 110 via wired and/or wireless communication connections. The computer device 112 is configured with (and/or has access to) image processing software and/or algorithms for analyzing the obtained images (discussed further with reference to FIGS. 2-4). For example, the image processing software and/or algorithms can identify and/or determine growth characteristics such as total number of growth rings, number of growth rings pith-to-bark, total RPI, RPI of inner portion (e.g., inner portions 140a, 140b, shown in FIG. 1B), RPI of outer portion (e.g., outer portions 142a, 142b, shown in FIG. 1B), pith location, pith symmetry, pith eccentricity, geometric center of the end surface, end surface diameter (e.g., SED D2, and/or LED D1), percent LW, LW/EW ratio, log density, or ovality, to name a few. The computer device 112 is further configured with processing software and/or algorithms for determining one or more mechanical properties (e.g., strength and stiffness) based at least on the primary growth characteristics (e.g., the ring count, pith location, etc.). For example, the computer device 112 can calculate a modulus of elasticity (MOE) and/or Young's Modulus based on two or more of the RPI, age (for which ring count is often a reliable indicator), or diameter of the end surface of the log 102. In some embodiments, the computer device 112 determines the one or more mechanical properties based on the secondary growth characteristics as described herein (e.g., the eccentricity E, the percent LW, etc.), and/or based on one or more supplemental characteristics as described herein (e.g., log sweep, log length, etc.).

The computer device 112 can include image processing software and/or algorithms configured to identify the growth characteristics (e.g., the primary growth characteristics and/or the secondary growth characteristics) of a single log (e.g., log 102) in an image that comprises a plurality of logs. For example, imaging devices 110a, 110b can capture one or more images of a plurality of logs from a pile of logs on a staging platform at a processing facility, and the computer device 112 can identify one or more of the individual logs (e.g., a target log) of the plurality of logs, identify the growth characteristics of each of the corresponding identified individual logs (including the target log), and determine one or more mechanical properties of the corresponding individual logs.

In some embodiments, supplemental characteristics of the log are obtained by one or more measurement devices 120, which can include an imaging device similar to those discussed herein, such as laser arrays, photo arrays, light field imaging devices, surface scanning devices, and cameras. In some embodiments, the supplemental characteristics include log age, end surface diameter (e.g., SED and/or LED), log sweep and/or bow, log size/length, number of logs, location of knot whorls and/or surface bulges, taper, or crook. In some embodiments, the supplemental characteristics are provided to the computer device 112, which is communicatively coupled to the measurement devices 120.

In some embodiments, supplemental characteristics, such as decay, cracks, or aspects of growth rings and the pith that are not, for example, visible via the first and/or second end surfaces 105, 104, of the log 102 are obtained via the one or more measurement devices 120. For example, the measurement devices 120 can include a computer tomography (CT) scanner configured to generate three-dimensional images of the log. As another example, the measurement devices 120 can include x-ray emitters and/or x-ray detectors configured to emit and/or detect x-rays, respectively. In some embodiments the x-rays detected by the x-ray detectors are processed to generate a CT scan including cross-sectional image data of the log 102. In some embodiments, the measurement devices 120 include one or more acoustic sensing devices configured to detect sound waves associated with the log 102. For example, sound waves can be passed through the log 102 and/or emitted by the log 102 and can be received by acoustic sensors of the measurement devices 120. The sound waves received by the acoustic sensors can be converted to sound signal data used to generate information (e.g., via processing software and/or algorithms configured to analyze sound data) about the internal supplemental characteristics of the log 102. In some embodiments, the measurement devices 120 are used to identify and/or measure the supplemental characteristics of at least a portion of the log 102.

In some embodiments, the system 100 includes a platform 130 configured to support and/or hold the log 102 while the image data is being obtained (e.g., via the imaging devices 110a, 110b). For example, the platform 130 can include a logging truck bed, a staging area, a conveyor belt, or other moving or stationary platform configured to support and/or hold one or more logs. One example advantage of the present technology is the ability to obtain image data, identify growth characteristics, and determine mechanical properties of one or more logs (including a plurality of logs) in a dynamic range of environments, including in the field immediately after harvesting (i.e., without even having to transport the log to a processing facility, and/or without having to unload the log from a logging truck bed).

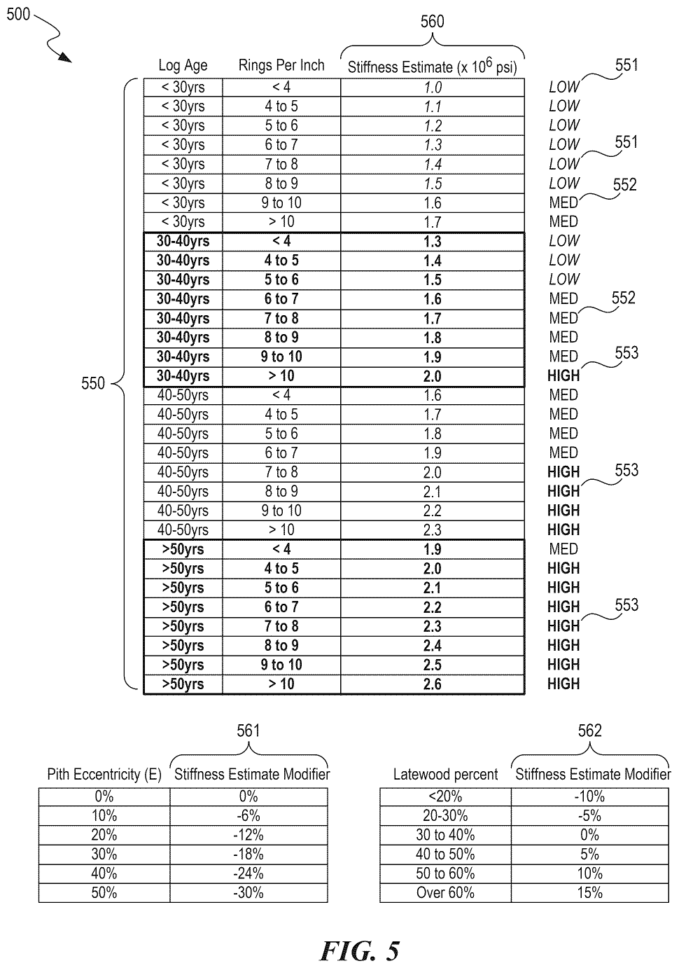

In some embodiments, the computer device 112 is configured to output a categorization of the log 102 (e.g., high stiffness, medium stiffness, and low stiffness) based on one or more of the primary and/or secondary growth characteristics, supplemental characteristics, and mechanical properties. In some embodiments, categorization of the log 102 includes tagging, marking, painting, or flagging the log 102. An example of such categorization is shown in FIG. 5.

In some embodiments the categorized logs are allocated for particular use based on their categorization. In some embodiments, allocating the log 102 includes physically transporting the log 102 to a bin, platform, or other area of a lumber yard or processing facility designated for such logs. In some embodiments allocation includes loading and/or shipping the log 102 to a destination configured to receive logs of the determined growth characteristics and/or mechanical properties. For example, logs receiving a categorization of high stiffness can be allocated to a first staging area designated for transport to construction-related uses, while logs receiving a categorization of medium stiffness can be allocated to a second staging area different from the first staging area, the second staging area designated for pulp production.

FIG. 1B is a partially-schematic view of the system 100 of FIG. 1A, in accordance with some embodiments of the present technology. In the present embodiments, the imaging device 110a is obtaining an image of the first end surface 105 of diameter D1 (i.e., the LED) along axis A1, and the imaging device 110b is obtaining an image of the second end surface 104 of diameter D2 along axis A2.

In some embodiments, the growth characteristics of each of the end surfaces 105, 104 are automatically identified and/or determined (e.g., by computer device 112) and compared to each other. This comparison is used to categorize the log 102, and/or used to determine the mechanical properties of the log 102. For example, as shown in FIG. 1B, the first end surface 105 can provide information on first growth characteristics corresponding to the first end surface 105, such as a first number of growth rings, a first total RPI, a first RPI of inner portion 140a, a first RPI of an outer portion 142a, a first pith location, etc., and the second end surface 104 can provide information on second growth characteristics corresponding to the second end surface 104, such as a second number of growth rings, a second total RPI, a second RPI of an inner portion 140b, a second RPI of an outer portion 142b, a second pith location, etc. The first and second growth characteristics can be analyzed to determine, for example, an average number of growth rings, an average RPI, an average pith location, etc., which can be used to categorize the log. Alternatively or in addition to this determination, mechanical properties corresponding to the determined growth characteristics of the first and second end surfaces 105, 104 can be determined, such as first and second strengths, stiffnesses, etc. These mechanical properties can be analyzed to provide, for example, an average strength, stiffness, etc. representative of an overall strength, stiffness, etc. of the log 102, which can then be used to categorize the log 102.

FIG. 2 is an illustration of an obtained image 200 of an end surface 205 of a log 202, in accordance with some embodiments of the present technology. In some embodiments, the image 200 is obtained (e.g., captured) by one or more of the imaging devices 110 described with reference to FIGS. 1A and/or 1B. In some embodiments, the image 200 is obtained from a database or storage medium (e.g., a cloud network, a computer storage medium, etc.). In the present embodiments, the image 200 includes a plurality of growth rings 230 extending radially outward from a pith 232 to a bark layer 234. For example, in the present embodiment, 18 growth rings are shown extending from the pith 232 to the bark layer 234.

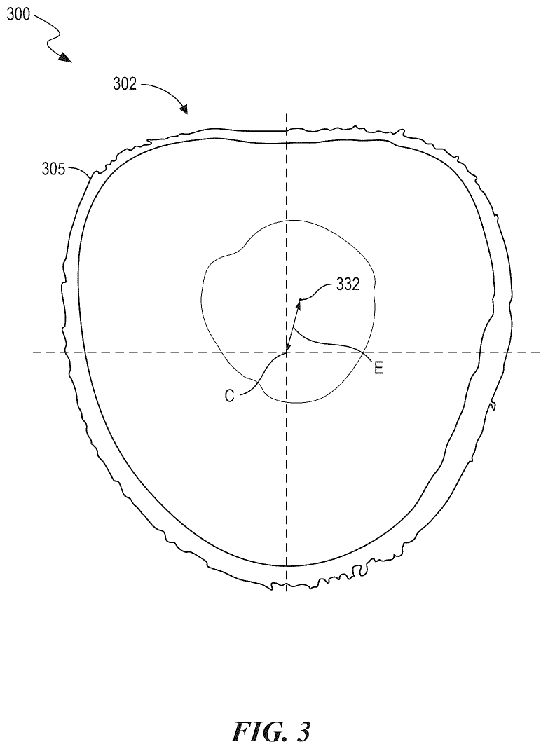

In some embodiments, a strength and/or stiffness of the log 202 is determined based on the plurality of growth rings 230 extending from the pith 232 to the bark layer 234. For example, stiffness can be calculated based on the RPI and ring count (RC) of the log using Formulas I and II below:

$$\begin{matrix}RPI = RC / ( D / 2 ) & ( Formula I ) \\ Stiffness = K 1 \times ( RPI ) + K 2 \times ( RC ) + F & ( Formula II )\end{matrix}$$

Where D is the diameter of the log and K1, K2, and F are empirically-determined constants. In some embodiments, one or more of the constants K1, K2, and F are determined based on analyses performed via one or more ML models, such as those provided in ML system 1000 described further with reference to FIG. 10. As described further with respect to FIGS. 3-5, in some embodiments, the stiffness can be adjusted and/or modified based on additional growth characteristics, such as eccentricity and LW/EW.

FIG. 3 is an illustration of an obtained image 300 of an end surface 305 of a log 302 in accordance with some embodiments of the present technology. In some embodiments, the image 300 is obtained (e.g., captured) by one or more of the imaging devices 110 described with reference to FIG. 1A. In some embodiments, the image 300 is obtained from a database or other storage medium. In the present embodiments, the image 300 includes a pith 332 offset from an approximate geometric center C of the log 302. The physical and mechanical properties (e.g., strength, stiffness etc.) of logs with high eccentricity are generally inferior to logs with low eccentricity.

The percent pith eccentricity or offset can be calculated based on the distance between the pith 332 and the approximate geometric center C of the log using Formula III below:

$$E ( \% ) = ( {E}_{c} / {d}_{s} ) \times 100 \% \qquad ( Formula III )$$

Where E_{c} is the distance between the pith 332 and the geometric center of the log C and dₛ is the smallest diameter of the log across the geometrical center C. In some embodiments, the determined eccentricity can be adjusted and/or modified based on one or more empirically-determined constants before being incorporated into a final stiffness calculation (discussed further with reference to FIG. 5). For example, eccentricity modified (Eₘ) can be determined using Formula IV below:

$${E}_{m} = E \times K 3 \qquad ( Formula IV )$$

In some embodiments, constant K3 is determined based on analyses performed via one or more ML models, such as those provided in ML system 1000 described further with reference to FIG. 10.

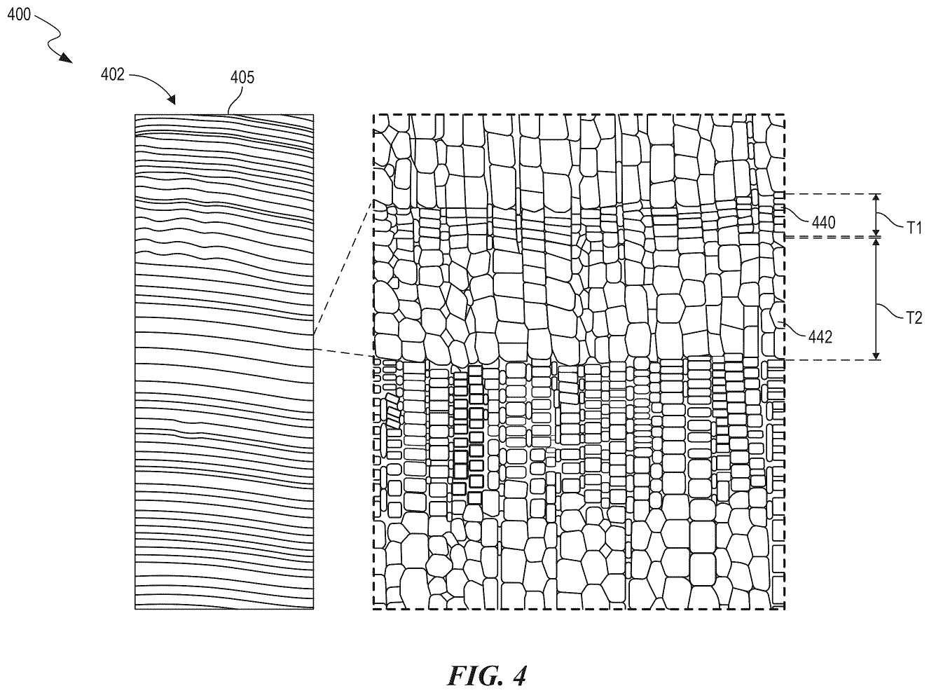

FIG. 4 is an illustration of an obtained image 400 of the latewood 440 (LW) and earlywood 442 (EW) of part of an end surface 405 of a log 402, in accordance with some embodiments of the present technology. In some embodiments, the image 400 is obtained (e.g., captured) by one or more of the imaging devices 110 described with reference to FIG. 1A. The physical and mechanical properties (e.g., the density) of logs with high proportions of LW (i.e., high percent LW and/or high LW/EW ratio) are generally superior to logs with low proportions of LW. In the present embodiments, FIG. 4 shows a growth ring of the end surface 405 with a LW 440 layer of thickness T1, and an EW 442 layer of thickness T2, where the thickness T1 of the LW 440 is less than the thickness T2 of the EW 442.

The density of log 402 can be calculated based on measuring the percent LW 440 and percent EW 442 of the end surface 405 and adjusting based on specific gravities of the latewood 440 and earlywood 442, using Formula V below:

$$Density = LW \% \times {SG}_{1 w} + EW \% \times {SG}_{ew} \qquad ( Formula V )$$

Where LW % is the measured percent LW, SG_{1w} is the specific gravity for LW, EW % is the measured percent EW, and SG_{ew} is the specific gravity for EW. In some embodiments, the specific gravity values are based on one or more empirically-determined values. In some embodiments, the specific gravity values are determined based on analyses performed via one or more ML models, such as those provided in ML system 1000 described further with reference to FIG. 10.

In some embodiments, the determined (e.g., measured and/or calculated) percent LW can be adjusted and/or modified based on one or more empirically-determined constants before being incorporated into a final stiffness calculation (discussed further with reference to FIG. 5). For example, percent LW modified (LWₘ) can be determined using Formula VI below:

$${LW}_{m} = LW \% \times K 4 \qquad ( Formula VI )$$

Where K4 is an empirically-determined constant. In some embodiments, constant K4 is determined based on analyses performed via one or more ML models, such as those provided in ML system 1000 described further with reference to FIG. 10.

FIG. 5 is a table 550 with log characteristics used for characterizations, in accordance with some embodiments of the present technology. In the present embodiments, a categorization scheme 500, represented via the table 550, includes a plurality of categorization thresholds 551, 552, 553 representative of high quality logs, medium quality logs, and low quality logs, respectively.

For example, table 550 shows how logs can be categorized based on (i) log age in years, divided into the following ranges: less than 30, 30-40, 40-50, and greater than 50, (ii) rings per inch (RPI), divided into the following ranges: less than 4, 4-5, 5-6, 6-7, 7-8, 8-9, 9-10, and greater than 10, for each of the log age ranges (i.e., less than 30, 30-40, 40-50, and greater than 50), and (iii) stiffness estimates of 1.0×10⁶-2.6×10⁶ pounds per square inch (psi) for each of the log age and RPI combinations.

As indicated in FIG. 5, a log less than 30 years old with less than 4 RPI can have a stiffness estimate of 1.0×10⁶ psi, and thus be categorized as a low quality log 551. A log less than 30 years old with 9-10 RPI can have a stiffness estimate of 1.6×10⁶ psi, and thus be categorized as a medium quality log 552. A log greater than 50 years old with 7-8 RPI can have a stiffness estimate of 2.3×10⁶ psi, and thus be categorized as a high quality log 553. As a further example, the high quality logs 551 can have a stiffness of 2.0×10⁶ psi or greater, the medium quality logs 552 can have a stiffness of between 1.6×10⁶-2.0×10⁶ psi, and the low quality logs 553 can have a stiffness of less than 1.6×10⁶ psi. In some embodiments, the high quality logs 551 have a stiffness of greater than 1.0, 1.1, 1.2, 1.3, 1.4, 1.5, 1.6, 1.7, 1.8, 1.9, or 2.0×10⁶ psi, or within a range of 1.0×10⁶-2.0×10⁶, or any increment therebetween (e.g., 1.85×10⁶ psi). In some embodiments, the medium quality logs 552 have a stiffness within a range of 1.0×10⁶-2.0×10⁶, or any increment therebetween (e.g., 1.2×10⁶-1.6×10⁶ psi). In some embodiments, the low quality logs have a stiffness of less than 1.0, 1.1, 1.2, 1.3, 1.4, 1.5, 1.6, 1.7, 1.8, 1.9, or 2.0×10⁶ psi, or any increment therebetween (e.g., 1.5×10⁵-9.5×10⁵ psi). In some embodiments, the high quality logs, 551, medium quality logs 552, and low quality logs 553 have none-overlapping categorization thresholds. In some embodiments one or more of the thresholds have at least some overlap. For example, the high quality logs 551 can have a stiffness of 2.0×10⁶ or greater, the medium quality logs can have a stiffness of 1.5×10⁶-2.1×10⁶, and the low quality logs can have a stiffness of less than 1.6×10⁶ psi. The characteristics shown in FIG. 5, including log age, rings per inch, pith eccentricity, and stiffness can be considered predetermined thresholds for ultimately categorizing logs into a particular category (e.g., high, medium, or low quality).

In some embodiments, the categorization scheme 500 is applied to one or more of the logs 102 of the system 100 that have had one or more of the end surfaces 104, 105 captured and/or imaged by the imaging device 110. In some embodiments the categorization scheme 500 is applied to one or more logs (e.g., the log 102) for which end surface images have been obtained from, for example, an online database or other storage medium. In some embodiments, the categorization scheme 500 is implemented using a computer device or system (e.g., the computer device 112 of FIGS. 1A and/or 1B and/or computer system 1200 of FIG. 12).

In some embodiments, the thresholds 551, 552, 553 are based on one or more mechanical properties of a given log. For example, the thresholds 551, 552, 553 can be based on log strength, stiffness, and the like.

In some embodiments, the mechanical properties are determined using one or more of the methods, analyses, and/or calculations described in this document. For example, the stiffness of the log can be determined using Formulas I and II, discussed further with reference to FIG. 2, based on the RPI and RC (i.e., age) of the log. As another example, the mechanical properties can be based on pith eccentricity E, which can be determined using Formula III, discussed further with reference to FIG. 3. As yet another example, the mechanical properties can be based on percent latewood LW %, which can be determined using Formula V, discussed further with reference to FIG. 4. In some embodiments, the mechanical properties are determined based on a combination of these features.

For example, an initial stiffness estimate 560 can be determined based on RPI and RC. Put another way, the initial stiffness estimate 560 can be determined based on the number of growth rings, the pith location, and/or the diameter of the log. The stiffness estimate 560 can then be modified and/or adjusted based on eccentricity E. Using Formulas III and IV, discussed further with reference to FIG. 3, a first stiffness adjustment 561 can be determined that factors eccentricity into the stiffness estimate 560. The stiffness estimate 560 can further be modified and/or adjusted based on percent latewood LW %. Using Formulas V and VI, discussed further with reference to FIG. 4, a second stiffness adjustment 562 can be determined that factors percent latewood LW % into the stiffness estimate 560. Accordingly, a final stiffness estimate can be calculated using Formula VII below:

$$Final Stiffness estimate = Initial Stiffness estimate \times {E}_{m} \times {LW}_{m} \qquad ( Formula VII )$$

Thus, a log that may not have a particularly high stiffness estimate based solely on growth rings and pith location can still be appropriately categorized as high quality if the log has correspondingly low pith eccentricity and high percent latewood LW %. Conversely, a log that has a high stiffness estimate based solely on growth rings and pith location can be appropriately categorized as medium or low quality if the log has correspondingly high pith eccentricity and low percent latewood LW %. For example, FIG. 5 shows a first stiffness adjustment 561 of between about 0% to −30% (i.e., a negative adjustment/modification to the initial stiffness estimate 560) based on an eccentricity of between about 0%-50%. In the present example, an eccentricity of 0% results in a first stiffness adjustment 561 of 0%, while an eccentricity of 50% results in a first stiffness adjustment 561 of −30%. As an additional example, FIG. 5 shows a second stiffness adjustment 562 of between about −10% to 15% (that is, negative 10% to positive 15%) based on a percent LW % of less than 20% to greater than 60%. In the present example, a percent LW % of less than 20% results in a second stiffness adjustment 562 of −10% (i.e., a negative adjustment/modification), while a percent LW % of greater than 60% results in a second stiffness adjustment 562 of 15% (i.e., a positive adjustment/modification).

In some embodiments, a categorical rule and/or threshold value associated with one or more of the growth characteristics is used to categorize the log. For example, logs that are less than 15 years old (i.e., juvenile wood, as determined, for example by ring count), can be automatically categorized as low quality and/or low stiffness logs. Logs that are greater than 15 years old (i.e., mature wood) can be further processed (e.g., via RPI and/or eccentricity determination) to determine whether the log is of medium or high quality.

In some embodiments, one or more supplemental characteristics (e.g., curve, number of knot whorls, etc.) can be used in a similar manner as discussed herein to determine the mechanical properties of the log and/or categorize the log based at least in part on the supplemental characteristics and/or determined mechanical properties. For example, a log with RPI and RC values corresponding with a categorization of high quality and/or high stiffness can be revised to a lower category (e.g., medium quality and/or stiffness) based on having greater than 5 knot whorls. In such an example, the presence of greater than 5 knot whorls may be a categorical threshold value that automatically reduces the category (i.e., from high to medium quality) of the log. Alternatively, the presence of greater than 5 knot whorls may be implemented as an adjustment and/or modifier to, for example, a stiffness value, similar to that shown for pith eccentricity or percent LW in FIG. 5 and as discussed herein.

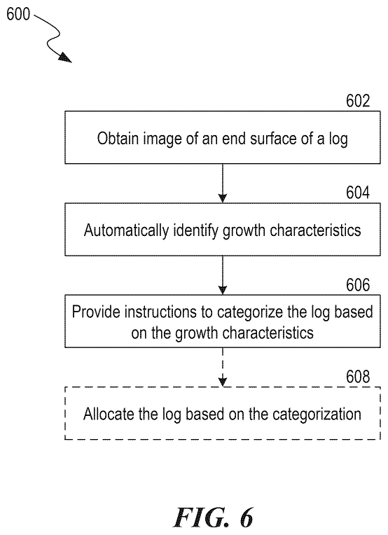

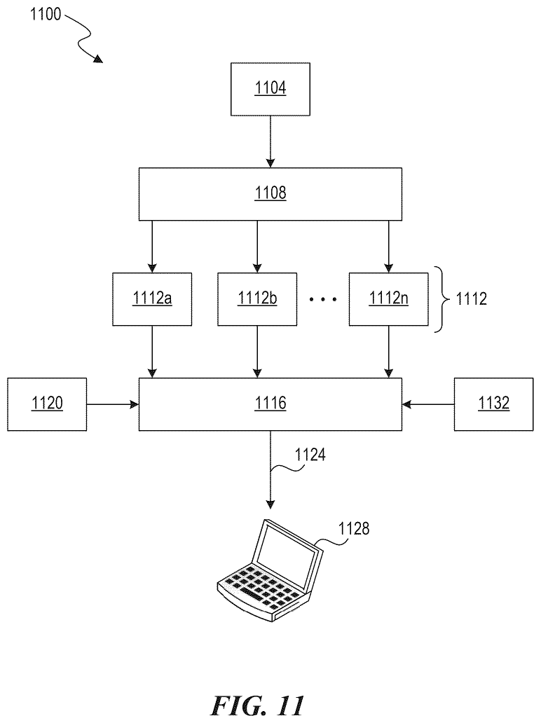

FIG. 6 is a method 600 of categorizing logs, in accordance with some embodiments of the present technology. In some embodiments, at least some of the blocks of method 600 are implemented using the system 100 and components of FIGS. 1A and/or 1B. In some embodiments, at least some of the blocks of method 600 are implemented using environment 1000 of FIG. 10, ML system 1100 of FIG. 11, and/or computer system 1200 of FIG. 1200.

At block 602, an image is obtained of an end surface of a log. In some embodiments, the image includes a pith and growth rings of the log. In some embodiments, the image is captured via one or more imaging devices, such as visible light cameras, hyperspectral imaging devices, and the like. In some embodiments, the image is obtained via an online or other computer-based storage medium, such as a cloud-based database. In some embodiments, obtaining the image of the end surface of the log includes imaging the end surface of the log along an axis that is at least approximately perpendicular to the end surface of the log.

At block 604, the growth characteristics of the end surface of the image are automatically identified using a computer. In some embodiments, the growth characteristics include information associated with the growth rings and/or the location of the pith. In some embodiments, the computer includes image processing software and/or image processing algorithms configured to automatically identify the growth characteristics (e.g., the growth rings and/or location of the pith) in the image. In some embodiments, the computer includes an ML system (e.g., ML system 1100 of FIG. 11) configured to automatically identify the growth characteristics in the image.

At block 606, instructions are provided to categorize the log based on the growth characteristics. Accordingly, in some embodiments, the log is categorized based on the instructions and/or based on the growth characteristics. For example, the log can be categorized as high quality, medium quality, or low quality, depending on the log's determined growth rings (e.g., ring count and/or RPI), and location of the pith. In some embodiments, the categorization includes marking and/or flagging the log, such as adding a paint marker to a surface of the log corresponding to the categorization.

At optional block 608, the log is allocated for a particular use based on the categorization. For example, high quality logs categorized in block 606 can be allocated to a conveyor belt or logging truck for further processing and/or delivery, medium quality logs can be allocated to a staging area for at least temporary storage, and low quality logs can be discarded.

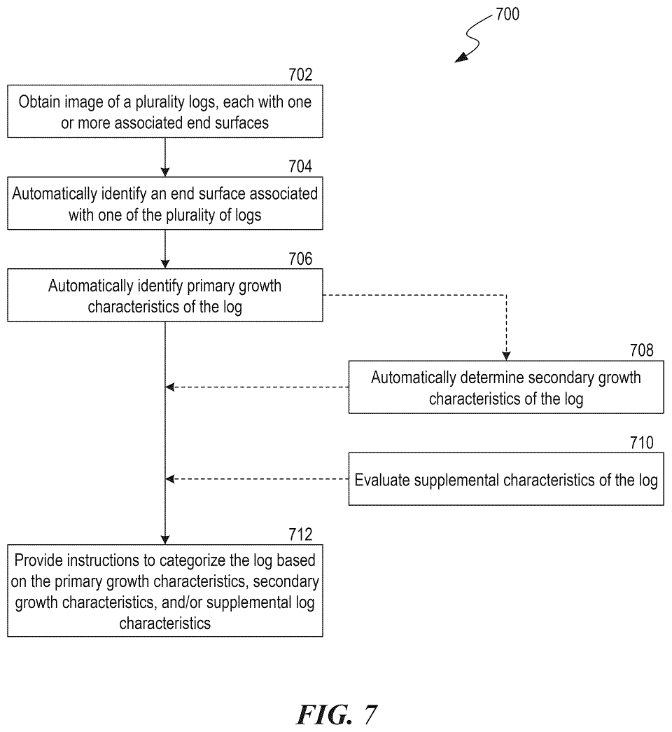

FIG. 7 is another method 700 of categorizing logs, in accordance with some embodiments of the present technology. In some embodiments, at least some of the blocks of method 700 are implemented using the system 100 and components of FIGS. 1A and/or 1B. In some embodiments, at least some of the blocks of method 700 are implemented using environment 1000 of FIG. 10, ML system 1100 of FIG. 11, and/or computer system 1200 of FIG. 1200.

At block 702, an image is obtained of a plurality of logs, where each log includes one or more associated end surfaces. For example, an image can be captured of a logging truck bed including a stack of logs, where the image includes at least portions of end surfaces corresponding to at least some of the logs.

At block 704, the end surface of one of the plurality of logs (referred to herein as the target log) is automatically identified. For example, the computer device 112 can include image processing software that automatically identifies the end surface of one or more of the logs on the truck bed, including the end surface of the target log.

At block 706, the primary growth characteristics (e.g., the number of growth rings and pith location) of the target log are automatically identified from the image. For example, the computer device includes image processing software and/or image processing algorithms configured to automatically identify the primary growth characteristics of the end surface of the target log in the image. In some embodiments, the computer includes an ML system configured to automatically identify the primary growth characteristics in the image.

At optional block 708, the secondary growth characteristics (e.g., the pith symmetry, pith eccentricity, LW/EW ratio, percent LW, percent EW, ovality) of the end surface of the target log are determined. For example, the computer device includes image processing software and/or image processing algorithms configured to automatically identify the secondary growth characteristics of the end surface of the target log in the image. In some embodiments, the computer includes an ML system configured to automatically identify the secondary growth characteristics in the image.

At optional block 710, the supplemental characteristics (e.g., log sweep, log length, size of knot whorls and/or surface bulges, location of knot whorls and/or surface bulges bow, taper, crook, decay, cracks) are evaluated. In some embodiments, the supplemental characteristics are first measured (e.g., via the measurement devices 120 of FIG. 1A) and provided to a computer device (e.g., the computer device 112) where software/algorithms are used to analyze and evaluate the supplemental characteristic data.

At block 712, instructions are provided to categorize the log based on the primary growth characteristics, secondary growth characteristics, and/or supplemental growth characteristics, depending on which characteristics were determined and/or evaluated. Accordingly, in some embodiments, the log is categorized based on the instructions, and/or based on the primary growth characteristics, secondary growth characteristics, and/or supplemental growth characteristics. For example, the log can be categorized as high quality, medium quality, or low quality, depending on the log's determined growth rings (e.g., primary growth characteristics), eccentricity (e.g., secondary growth characteristics), and sweep (e.g., supplemental characteristics).

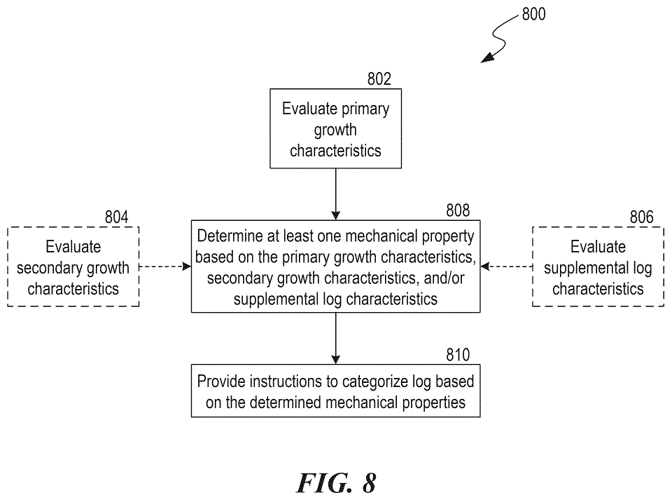

FIG. 8 is yet another method 800 of categorizing logs, in accordance with some embodiments of the present technology. In some embodiments, at least some of the blocks of method 800 are implemented using the system 100 and components of FIGS. 1A and/or 1B. In some embodiments, at least some of the blocks of method 800 are implemented using environment 1000 of FIG. 10, ML system 1100 of FIG. 11, and/or computer system 1200 of FIG. 1200.

At blocks 802, 804, and 806, the primary growth characteristics (shown in block 802), optional secondary growth characteristics (shown in block 804), and optional supplemental log characteristics (shown in block 806) are evaluated and/or analyzed. In some embodiments, the primary and secondary growth characteristics and/or supplemental characteristics are evaluated automatically via a computer device (e.g., via software and/or algorithms) based on obtained image data of an end surface of a log (in the case of the primary and secondary growth characteristics) or measured supplemental data (in the case of the supplemental characteristics.

At block 808 at least one mechanical property of the log is determined based on the evaluated and/or analyzed primary growth characteristics, secondary growth characteristics, or supplemental characteristics. For example, based on the identified growth rings and location of the pith, RPI and RC values can be determined. Using Formulas I and II of FIG. 2, stiffness can be determined. In some embodiments, the RPI and RC determinations are performed automatically by the computer. In some embodiments, the mechanical properties include one or more of stiffness and strength.

At block 810, instructions are provided to categorize the log based on the determined mechanical properties. Accordingly, in some embodiments, the log is categorized based on the instructions and/or based on the determined mechanical properties. For example, the log can be categorized as high quality, medium quality, or low quality, depending on the log's determined (and, in some embodiments, adjusted/modified) stiffness.

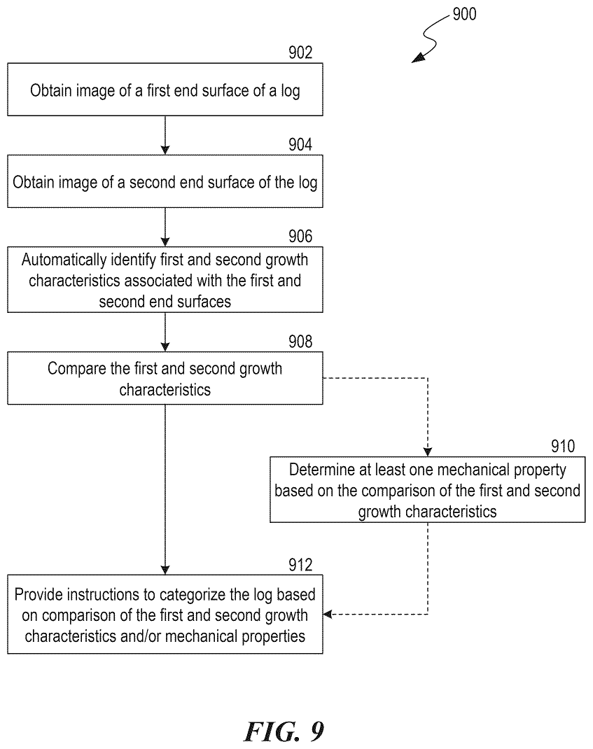

FIG. 9 is still another method 900 of categorizing logs, in accordance with some embodiments of the present technology. In some embodiments, at least some of the blocks of method 900 are implemented using the system 100 and components of FIGS. 1A and/or 1B. In some embodiments, at least some of the blocks of method 900 are implemented using environment 1000 of FIG. 10, ML system 1100 of FIG. 11, and/or computer system 1200 of FIG. 1200.

At block 902, a first image of a first end surface of a log is obtained. For example, a first imaging device (e.g., imaging device 110a of FIGS. 1A and 1B) can capture a first image of the end surface of a log corresponding to the LED. At block 904, a second image of a second end surface of the log is obtained. For example, a second imaging device (e.g., imaging device 110b) can capture a second image of the end surface of the log corresponding to the SED. In some embodiments, the first and second images are obtained near-simultaneously, increasing efficiency of subsequent comparisons of the two images, as discussed further herein.

At block 906, the first and second growth characteristics corresponding to the first and second end surfaces of the first and second images are automatically identified (e.g., via computer device 112 using image processing software. At block 908, the first and second growth characteristics are compared. For example, the ring count of the first end surface can be compared to the ring count of the second end surface, the pith location of the first end surface can be compared to the pith location of the second end surface, etc. In some embodiments, the comparison is performed automatically by the computer device.

At optional block 910, at least one mechanical property is determined based on the comparison of the first and second growth characteristics. For example, based on an average of the ring counts and pith location between the LED and the SED, a corresponding stiffness can be determined for the log.

At block 912, instructions are provided to categorize the log based on the comparison of the first and second growth characteristics and/or based on the determined mechanical properties. Accordingly, in some embodiments, the log is categorized based on the instructions and/or based on the comparison of the first and second growth characteristics, and/or based on the determined mechanical properties.

### Example Computing, Network, and Machine Learning Environments

FIG. 10 is a system diagram illustrating an example of a computing environment 1000 in which the disclosed system operates in some embodiments. In some embodiments, environment 1000 includes one or more client computing devices 1005A-D, examples of which can host the computer system 1200 discussed further with reference to FIG. 12, and/or ML system 1100, discussed further with reference to FIG. 11. Client computing devices 1005 operate in a networked environment using logical connections through network 1030 to one or more remote computers, such as a server computing device.

In some embodiments, server 1010 is an edge server which receives client requests and coordinates fulfillment of those requests through other servers, such as servers 1020A-C. In some embodiments, server computing devices 1010 and 1020 comprise computing systems, such as the system 1200. Though each server computing device 1010 and 1020 is displayed logically as a single server, server computing devices can each be a distributed computing environment encompassing multiple computing devices located at the same or at geographically disparate physical locations. In some embodiments, each server 1020 corresponds to a group of servers.

Client computing devices 1005 and server computing devices 1010 and 1020 can each act as a server or client to other server or client devices. In some embodiments, servers (1010, 1020A-C) connect to a corresponding database (1015, 1025A-C). As discussed above, each server 1020 can correspond to a group of servers, and each of these servers can share a database or can have its own database. Databases 1015 and 1025 warehouse (e.g., store) information such as tree species, log harvest location, climate data, weather data, log age, time elapsed since harvest, growth ring characteristics, pith characteristics, geometric center of the log end surface, pith eccentricity, percent LW, LW/EW, SED, LED, log length, log sweep and/or bow, size/shape/number/arrangement of knot whorls and/or surface bulges, strength, stiffness, crook, taper, ovality, and so on. Though databases 1015 and 1025 are displayed logically as single units, databases 1015 and 1025 can each be a distributed computing environment encompassing multiple computing devices, can be located within their corresponding server, or can be located at the same or at geographically disparate physical locations.

Network 1030 can be a local area network (LAN) or a wide area network (WAN), but can also be other wired or wireless networks. In some embodiments, network 1030 is the Internet or some other public or private network. Client computing devices 1005 are connected to network 1030 through a network interface, such as by wired or wireless communication. While the connections between server 1010 and servers 1020 are shown as separate connections, these connections can be any kind of local, wide area, wired, or wireless network, including network 1030 or a separate public or private network.

FIG. 11 is a block diagram illustrating an example ML system 1100, in accordance with some embodiments. The ML system 1100 is implemented using components of the example computer system 1200 illustrated and described in more detail with reference to FIG. 12. Different embodiments of the ML system 1100 include different and/or additional components and are connected in different ways. The ML system 1100 is sometimes referred to as a ML module.

The ML system 1100 includes a feature extraction module 1108 implemented using components of the example computer system 1200 illustrated and described in more detail with reference to FIG. 12. In some embodiments, the feature extraction module 1108 extracts a feature vector 1112 from input data 1104. For example, the input data 1104 includes tree species, log harvest location, climate data, weather data, log age, time elapsed since harvest, growth ring characteristics, pith characteristics, geometric center of the log end surface, pith eccentricity, percent LW, LW/EW, SED, LED, log length, log sweep and/or bow, size/shape/number/arrangement of knot whorls and/or surface bulges, crook, taper, ovality, and the like. The feature vector 1112 includes features 1112a, 1112b, . . . , 1112n. The feature extraction module 1108 reduces the redundancy in the input data 1104, for example, repetitive data values, to transform the input data 1104 into the reduced set of features 1112, for example, features 1112a, 1112b, . . . , 1112n. The feature vector 1112 contains the relevant information from the input data 1104, such that events or data value thresholds of interest are identified by the ML model 1116 by using a reduced representation. In some example embodiments, the following dimensionality reduction techniques are used by the feature extraction module 1108: independent component analysis, Isomap, kernel principal component analysis (PCA), latent semantic analysis, partial least squares, PCA, multifactor dimensionality reduction, nonlinear dimensionality reduction, multilinear PCA, multilinear subspace learning, semidefinite embedding, autoencoder, and deep feature synthesis.

In alternate embodiments, the ML model 1116 performs deep learning (also known as deep structured learning or hierarchical learning) directly on the input data 1104 to learn data representations, as opposed to using task-specific algorithms. In deep learning, no explicit feature extraction is performed; the features 1112 are implicitly extracted by the ML system 1100. For example, the ML model 1116 uses a cascade of multiple layers of nonlinear processing units for implicit feature extraction and transformation. Each successive layer uses the output from the previous layer as input. The ML model 1116 thus learns in supervised (e.g., classification) and/or unsupervised (e.g., pattern analysis) modes. The ML model 1116 learns multiple levels of representations that correspond to different levels of abstraction, wherein the different levels form a hierarchy of concepts. The multiple levels of representation configure the ML model 1116 to differentiate features of interest from background features.

In alternative example embodiments, the ML model 1116, for example, in the form of a CNN generates the output 1124, without the need for feature extraction, directly from the input data 1104. The output 1124 is provided to the computer device 1128. The computer device 1128 is a server, computer, tablet, smartphone, etc., implemented using components of the example computer system 1200 illustrated and described in more detail with reference to FIG. 12. In some embodiments, the steps performed by the ML system 1100 are stored in memory on the computer device 1128 for execution. In other embodiments, the output 1124 is displayed on electronic displays of the computer device 1128.

A CNN is a type of feed-forward artificial neural network in which the connectivity pattern between its neurons is inspired by the organization of a visual cortex. Individual cortical neurons respond to stimuli in a restricted area of space known as the receptive field. The receptive fields of different neurons partially overlap such that they tile the visual field. The response of an individual neuron to stimuli within its receptive field is approximated mathematically by a convolution operation. CNNs are based on biological processes and are variations of multilayer perceptrons designed to use minimal amounts of preprocessing.

In embodiments, the ML model 1116 is a CNN that includes both convolutional layers and max pooling layers. For example, the architecture of the ML model 1116 is “fully convolutional,” which means that variable sized sensor data vectors are fed into it. For convolutional layers, the ML model 1116 specifies a kernel size, a stride of the convolution, and an amount of zero padding applied to the input of that layer. For the pooling layers, the model 1116 specifies the kernel size and stride of the pooling.

In some embodiments, the ML system 1100 trains the ML model 1116, based on the training data 1120, to correlate the feature vector 1112 to expected outputs in the training data 1120. As part of the training of the ML model 1116, the ML system 1100 forms a training set of features and training labels by identifying a positive training set of features that have been determined to have a desired property in question, and, in some embodiments, forms a negative training set of features that lack the property in question.

The ML system 1100 applies ML techniques to train the ML model 1116, that when applied to the feature vector 1112, outputs indications of whether the feature vector 1112 has an associated desired property or properties, such as a probability that the feature vector 1112 has a particular Boolean property, or an estimated value of a scalar property. In embodiments, the ML system 1100 further applies dimensionality reduction (e.g., via linear discriminant analysis (LDA), PCA, or the like) to reduce the amount of data in the feature vector 1112 to a smaller, more representative set of data.

In embodiments, the ML system 1100 uses supervised ML to train the ML model 1116, with feature vectors of the positive training set and the negative training set serving as the inputs. In some embodiments, different ML techniques, such as linear support vector machine (linear SVM), boosting for other algorithms (e.g., AdaBoost), logistic regression, naïve Bayes, memory-based learning, random forests, bagged trees, decision trees, boosted trees, boosted stumps, neural networks, CNNs, etc., are used. In some example embodiments, a validation set 1132 is formed of additional features, other than those in the training data 1120, which have already been determined to have or to lack the property in question. The ML system 1100 applies the trained ML model 1116 to the features of the validation set 1132 to quantify the accuracy of the ML model 1116. Common metrics applied in accuracy measurement include Precision and Recall, where Precision refers to a number of results the ML model 1116 correctly predicted out of the total it predicted, and Recall is a number of results the ML model 1116 correctly predicted out of the total number of features that had the desired property in question. In some embodiments, the ML system 1100 iteratively re-trains the ML model 1116 until the occurrence of a stopping condition, such as the accuracy measurement indication that the ML model 1116 is sufficiently accurate, or a number of training rounds having taken place. In embodiments, the validation set 1132 includes data corresponding to confirmed mechanical properties and/or weightings/constants and combinations thereof. This allows the detected values to be validated using the validation set 1132. The validation set 1132 is generated based on the analysis to be performed.

FIG. 12 is a block diagram illustrating an example computer system 1200, in accordance with some embodiments. Components of the example computer system 1200 are used to implement one or more portions of method 600 of FIG. 6, method 700 of FIG. 7, method 800 of FIG. 8, method 900 of FIG. 9, and/or perform analyses and calculations described throughout his document. In some embodiments, components of the example computer system 1200 are used to implement the ML system 1100 illustrated and described in more detail with reference to FIG. 11. At least some operations described herein are implemented on the computer system 1200.

The computer system 1200 includes one or more central processing units (“processors”) 1202, main memory 1206, non-volatile memory 1210, network adapters 1212 (e.g., network interface), video displays 1218, input/output devices 1220, control devices 1222 (e.g., keyboard and pointing devices), drive units 1224 including a storage medium 1226, and a signal generation device 1220 that are communicatively connected to a bus 1216. The bus 1216 is illustrated as an abstraction that represents one or more physical buses and/or point-to-point connections that are connected by appropriate bridges, adapters, or controllers. In embodiments, the bus 1216, includes a system bus, a Peripheral Component Interconnect (PCI) bus or PCI-Express bus, a HyperTransport or industry standard architecture (ISA) bus, a small computer system interface (SCSI) bus, a universal serial bus (USB), IIC (I2C) bus, or an Institute of Electrical and Electronics Engineers (IEEE) standard 1094 bus (also referred to as “Firewire”).

In embodiments, the computer system 1200 shares a similar computer processor architecture as that of a desktop computer, tablet computer, personal digital assistant (PDA), mobile phone, game console, music player, wearable electronic device (e.g., a watch), network-connected (“smart”) device (e.g., a television or home assistant device), virtual/augmented reality systems (e.g., a head-mounted display), or another electronic device capable of executing a set of instructions (sequential or otherwise) that specify action(s) to be taken by the computer system 1200.

While the main memory 1206, non-volatile memory 1210, and storage medium 1226 (also called a “machine-readable medium”) are shown to be a single medium, the term “machine-readable medium” and “storage medium” should be taken to include a single medium or multiple media (e.g., a centralized/distributed database and/or associated caches and servers) that store one or more sets of instructions 1228. The term “machine-readable medium” and “storage medium” shall also be taken to include any medium that is capable of storing, encoding, or carrying a set of instructions for execution by the computer system 1200.

In general, the routines executed to implement the embodiments of the disclosure are implemented as part of an operating system or a specific application, component, program, object, module, or sequence of instructions (collectively referred to as “computer programs”). The computer programs typically include one or more instructions (e.g., instructions 1204, 1208, 1228) set at various times in various memory and storage devices in a computer device. When read and executed by the one or more processors 1202, the instruction(s) cause the computer system 1200 to perform operations to execute elements involving the various aspects of the disclosure.

Moreover, while embodiments have been described in the context of fully functioning computer devices, those skilled in the art will appreciate that the various embodiments are capable of being distributed as a program product in a variety of forms. The disclosure applies regardless of the particular type of machine or computer-readable media used to actually effect the distribution.

Further examples of machine-readable storage media, machine-readable media, or computer-readable media include recordable-type media such as volatile and non-volatile memory devices 1210, floppy and other removable disks, hard disk drives, optical discs (e.g., Compact Disc Read-Only Memory (CD-ROMS), Digital Versatile Discs (DVDs)), and transmission-type media such as digital and analog communication links.

The network adapter 1212 enables the computer system 1200 to mediate data in a network 1214 with an entity that is external to the computer system 1200 through any communication protocol supported by the computer system 1200 and the external entity. In embodiments, the network adapter 1212 includes a network adapter card, a wireless network interface card, a router, an access point, a wireless router, a switch, a multilayer switch, a protocol converter, a gateway, a bridge, a bridge router, a hub, a digital media receiver, and/or a repeater.

In embodiments, the network adapter 1212 includes a firewall that governs and/or manages permission to access proxy data in a computer network and tracks varying levels of trust between different machines and/or applications. In embodiments, the firewall is any number of modules having any combination of hardware and/or software components able to enforce a predetermined set of access rights between a particular set of machines and applications, machines and machines, and/or applications and applications (e.g., to regulate the flow of traffic and resource sharing between these entities). The firewall additionally manages and/or has access to an access control list that details permissions including the access and operation rights of an object by an individual, a machine, and/or an application, and the circumstances under which the permission rights stand.

In embodiments, the functions performed in the processes and methods are implemented in differing order. Furthermore, the outlined steps and operations are only provided as examples. For example, some of the steps and operations are optional, combined into fewer steps and operations, or expanded into additional steps and operations without detracting from the essence of the disclosed embodiments.

In embodiments, the techniques introduced here are implemented by programmable circuitry (e.g., one or more microprocessors), software and/or firmware, special-purpose hardwired (i.e., non-programmable) circuitry, or a combination of such forms. In embodiments, special-purpose circuitry is in the form of one or more application-specific integrated circuits (ASICs), programmable logic devices (PLDs), field-programmable gate arrays (FPGAs), etc.

### CONCLUSION

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof means any connection or coupling, either direct or indirect, between two or more elements; the coupling or connection between the elements can be physical, logical, or a combination thereof. Additionally, the words “herein,” “above,” “below,” and words of similar import, when used in this application, refer to this application as a whole and not to any particular portions of this application. Where the context permits, words in the above Detailed Description using the singular or plural number may also include the plural or singular number, respectively. The word “or” in reference to a list of two or more items, covers all of the following interpretations of the word: any of the items in the list, all of the items in the list, and any combination of the items in the list.

The above Detailed Description of examples of the technology is not intended to be exhaustive or to limit the technology to the precise form disclosed above. While specific examples for the technology are described above for illustrative purposes, various equivalent modifications are possible within the scope of the technology, as those skilled in the relevant art will recognize. For example, while processes or blocks are presented in a given order, alternative embodiments may perform routines having steps, or employ systems having blocks, in a different order, and some processes or blocks may be deleted, moved, added, subdivided, combined, and/or modified to provide alternative or sub-combinations. Each of these processes or blocks may be implemented in a variety of different ways. Also, while processes or blocks are at times shown as being performed in series, these processes or blocks may instead be performed or implemented in parallel, or may be performed at different times. Further, any specific numbers noted herein are only examples: alternative embodiments may employ differing values or ranges.

The teachings of the technology provided herein can be applied to other systems, not necessarily the system described above. The elements and acts of the various examples described above can be combined to provide further embodiments of the technology. Some alternative embodiments of the technology may include not only additional elements to those embodiments noted above, but also may include fewer elements.

These and other changes can be made to the technology in light of the above Detailed Description. While the above description describes certain examples of the technology, and describes the best mode contemplated, no matter how detailed the above appears in text, the technology can be practiced in many ways. Details of the system may vary considerably in its specific implementation, while still being encompassed by the technology disclosed herein. As noted above, specific terminology used when describing certain features or aspects of the technology should not be taken to imply that the terminology is being redefined herein to be restricted to any specific characteristics, features, or aspects of the technology with which that terminology is associated. In general, the terms used in the following claims should not be construed to limit the technology to the specific examples disclosed in the specification, unless the above Detailed Description section explicitly defines such terms. Accordingly, the actual scope of the technology encompasses not only the disclosed examples, but also all equivalent ways of practicing or implementing the technology under the claims.

To reduce the number of claims, certain aspects of the technology are presented below in certain claim forms, but the applicant contemplates the various aspects of the technology in any number of claim forms. For example, while only one aspect of the technology is recited as a computer-readable medium claim, other aspects may likewise be embodied as a computer-readable medium claim, or in other forms, such as being embodied in a means-plus-function claim. Any claims intended to be treated under 35 U.S.C. § 212(f) will begin with the words “means for,” but use of the term “for” in any other context is not intended to invoke treatment under 35 U.S.C. § 212(f). Accordingly, the applicant reserves the right to pursue additional claims after filing this application to pursue such additional claim forms, in either this application or in a continuing application.

The present technology is illustrated, for example, according to various aspects described below as numbered clauses or embodiments (1, 2, 3, etc.) for convenience. These are provided as examples and do not limit the present technology. It is noted that any of the dependent clauses can be combined in any combination and placed into a respective independent clause.
1. A method of categorizing a log, the method comprising:
obtaining an image of an end surface of a log, wherein the end surface includes a pith and growth rings;
automatically identifying one or more growth characteristics of the log based on the pith and the growth rings; and
providing instructions to categorize the log based on the identified growth characteristics.
2. The method of any one of the clauses herein, wherein the identified growth characteristics include at least two of age, end surface diameter, and growth rings per inch (RPI).
3. The method of any one of the clauses herein, wherein the identified growth characteristics includes a percent pith eccentricity.
4. The method of any one of the clauses herein, further comprising determining a modulus of elasticity (MOE) based on the identified growth characteristics, and wherein providing instructions comprises providing first instructions to categorize the log into a first category if the MOE is at least equal to a predetermined threshold or providing second instructions to categorize the log into a second category if the MOE is less than the predetermined threshold.
5. The method of any one of the clauses herein, wherein the identified growth characteristics include ring count, geometric center of the end surface, location of the pith, latewood-to-earlywood ratio (LW/EW), and/or percent latewood (LW %).
6. The method of any one of the clauses herein, further comprising determining a stiffness of the log based on the identified growth characteristics, wherein categorizing the log is based at least in part on the determined stiffness.
7. The method of any one of the clauses herein, wherein providing instructions to categorize the log comprises:
providing first instructions to categorize the log in a first category if the stiffness is at least 1.6×10⁶ pounds per square inch (psi); and
providing second instructions to categorize the log in a second category if the stiffness is less than 1.6×10⁶ psi.
8. The method of any one of the clauses herein, wherein the end surface is a first end surface, the growth rings are first growth rings, and the first end surface includes a first diameter, the method further comprising:
obtaining an image of an opposing second end surface of the log, wherein the second end surface includes second growth rings and a second diameter different than the first diameter;
automatically identifying an age of the log based on a difference between at least one of (i) the first diameter and the second diameter or (ii) the first growth rings and the second growth rings; and
providing further instructions to categorize the log based on the identified age of the log.
9. The method of any one of the clauses herein, further comprising:
automatically determining a percent pith eccentricity of the end surface of the log based on a location of a pith relative to a geometric center of the end surface of the log; and
updating the instructions to categorize the log based on the percent pith eccentricity.
10. The method of any of the clauses herein, wherein the end surface is a first end surface, the pith is a first pith, and the first end surface includes a first geometric center, the method further comprising:
obtaining an image of an opposing second end surface of the log, wherein the second end surface includes a second pith and a second geometric center;
determining a first percent pith eccentricity based on a location of the first pith relative to the first geometric center;
determining a second percent pith eccentricity based on a location of the second pith relative to the second geometric center; and
updating the instructions to categorize the log based on the first pith eccentricity and the second pith eccentricity.
11. The method of any one of the clauses herein, further comprising:
automatically determining a percent latewood of the end surface of the log based on the growth rings of the log; and
updating the instructions to categorize the log based on the percent latewood of the end surface of the log.
12. The method of any of the clauses herein, further comprising evaluating one or more supplemental characteristics of the log, wherein the one or more supplemental characteristics include at least one of log sweep, log length, size of knot whorls, or location of knot whorls.
13. The method of any of the clauses herein, wherein obtaining the image includes capturing the image via a hyperspectral camera.
14. The method of any of the clauses herein, wherein the log has a moisture content of at least 30% by weight based on oven dry weight of the log.
15. The method of any of the clauses herein, wherein a time elapsed since harvesting the log is greater than seven days.
16. A system of categorizing logs, the system comprising:
a platform configured to hold a log;
an imaging device positioned to capture an image of an end surface of the log;
a processor; and
at least one non-transitory memory storing instructions which, when executed by the processor, cause the system to:
   obtain, via the imaging device, an image of the end surface of the log;
   automatically identify one or more growth characteristics of the log based on the obtained image; and
   provide instructions to categorize the log into one of multiple categories based on the identified growth characteristics.
17. The system of any one of the clauses herein, wherein the identified growth characteristics include end surface diameter, age, and growth rings per inch (RPI), or percent pith eccentricity.
18. The system of any one of the clauses herein, wherein the imaging device is a first imaging device, the image is a first image, and the end surface is a first end surface, the system further comprising a second imaging device positioned to capture a second image of a second end surface of the log.
19. The system of clause 18, wherein the memory further causes the system to:
obtain, via the second imaging device, the second image of the second end surface of the log; automatically identify an age of the log based on the first image and the second image; and update the instructions to categorize the log based on the identified age of the log.
20. The system of any one of the clauses herein, further comprising a computer tomography (CT) scanner configured to generate a three-dimensional image of the log, wherein the memory further causes the system to obtain a supplemental characteristic of the log based on the three-dimensional image, and wherein providing instructions to categorize the log is based on the obtained supplemental characteristic.
21. The system of any one of the clauses herein, wherein the memory further causes the system to determine at least one mechanical property based on the growth characteristics, wherein the at least one mechanical property includes stiffness, and the instructions are based at least in part on the at least one mechanical property.
22. The system of any one of the clauses herein, wherein the log is a first log, the image is a first image, the platform is configured to hold the first log and a second log, and the imaging device is configured to capture one or more images including the first end surface of the first log and a second end surface of the second log, and wherein the memory further causes the system to:
obtain, via the imaging device, a second image of an end surface of the second log;
automatically identify growth characteristics of the second log based on the second image; and
provide instructions to categorize the second based on the growth characteristics of the second log.

We claim:

1. A method of categorizing a log, the method comprising:
obtaining an image of an end surface of a log, wherein the end surface includes a pith and growth rings;
automatically identifying one or more growth characteristics of the log based on the pith and the growth rings; and
providing instructions to categorize the log based on the identified growth characteristics.

2. The method of claim 1 wherein the identified growth characteristics include at least two of age, end surface diameter, and growth rings per inch (RPI).

3. The method of claim 2, wherein the identified growth characteristics includes a percent pith eccentricity.

4. The method of claim 2, further comprising determining a modulus of elasticity (MOE) based on the identified growth characteristics, and wherein providing instructions comprises providing first instructions to categorize the log into a first category if the MOE is at least equal to a predetermined threshold or providing second instructions to categorize the log into a second category if the MOE is less than the predetermined threshold.

5. The method of claim 1 wherein the identified growth characteristics include ring count, geometric center of the end surface, location of the pith, latewood-to-earlywood ratio (LW/EW), and/or percent latewood (LW %).

6. The method of claim 1, further comprising determining a stiffness of the log based on the identified growth characteristics, wherein categorizing the log is based at least in part on the determined stiffness.

7. The method of claim 6, wherein providing instructions to categorize the log comprises:
providing first instructions to categorize the log in a first category if the stiffness is at least 1.6×10⁶ pounds per square inch (psi); and
providing second instructions to categorize the log in a second category if the stiffness is less than 1.6×10⁶ psi.

8. The method of claim 1 wherein the end surface is a first end surface, the growth rings are first growth rings, and the first end surface includes a first diameter, the method further comprising:
obtaining an image of an opposing second end surface of the log, wherein the second end surface includes second growth rings and a second diameter different than the first diameter;
automatically identifying an age of the log based on a difference between at least one of (i) the first diameter and the second diameter or (ii) the first growth rings and the second growth rings; and
providing further instructions to categorize the log based on the identified age of the log.

9. The method of claim 1, further comprising:
automatically determining a percent pith eccentricity of the end surface of the log based on a location of a pith relative to a geometric center of the end surface of the log; and
updating the instructions to categorize the log based on the percent pith eccentricity.

10. The method of claim 1 wherein the end surface is a first end surface, the pith is a first pith, and the first end surface includes a first geometric center, the method further comprising:
obtaining an image of an opposing second end surface of the log, wherein the second end surface includes a second pith and a second geometric center;
determining a first percent pith eccentricity based on a location of the first pith relative to the first geometric center;
determining a second percent pith eccentricity based on a location of the second pith relative to the second geometric center; and
updating the instructions to categorize the log based on the first pith eccentricity and the second pith eccentricity.

11. The method of claim 1, further comprising:
automatically determining a percent latewood of the end surface of the log based on the growth rings of the log; and
updating the instructions to categorize the log based on the percent latewood of the end surface of the log.

12. The method of claim 1, further comprising evaluating one or more supplemental characteristics of the log, wherein the one or more supplemental characteristics include at least one of log sweep, log length, size of knot whorls, or location of knot whorls.

13. The method of claim 1 wherein obtaining the image includes capturing the image via a hyperspectral camera.

14. The method of claim 1 wherein the log has a moisture content of at least 30% by weight based on oven dry weight of the log.

15. The method of claim 1 wherein a time elapsed since harvesting the log is greater than seven days.

16. A system of categorizing logs, the system comprising:
a platform configured to hold a log;
an imaging device positioned to capture an image of an end surface of the log;
a processor; and
at least one non-transitory memory storing instructions which, when executed by the processor, cause the system to:
obtain, via the imaging device, an image of the end surface of the log;
automatically identify one or more growth characteristics of the log based on the obtained image; and
provide instructions to categorize the log into one of multiple categories based on the identified growth characteristics.

17. The system of claim 16 wherein the identified growth characteristics include end surface diameter, age, and growth rings per inch (RPI), or percent pith eccentricity.

18. The system of claim 16 wherein the imaging device is a first imaging device, the image is a first image, and the end surface is a first end surface, the system further comprising a second imaging device positioned to capture a second image of a second end surface of the log.

19. The system of claim 18, wherein the memory further causes the system to:
obtain, via the second imaging device, the second image of the second end surface of the log;
automatically identify an age of the log based on the first image and the second image; and
update the instructions to categorize the log based on the identified age of the log.

20. The system of claim 16, further comprising a computer tomography (CT) scanner configured to generate a three-dimensional image of the log, wherein the memory further causes the system to obtain a supplemental characteristic of the log based on the three-dimensional image, and wherein providing instructions to categorize the log is based on the obtained supplemental characteristic.

21. The system of claim 16 wherein the memory further causes the system to determine at least one mechanical property based on the growth characteristics, wherein the at least one mechanical property includes stiffness, and the instructions are based at least in part on the at least one mechanical property.

22. The system of claim 16 wherein the log is a first log, the end surface is a first end surface, the image is a first image, the platform is configured to hold the first log and a second log, and the imaging device is configured to capture one or more images including the first end surface of the first log and a second end surface of the second log, and wherein the memory further causes the system to:
obtain, via the imaging device, a second image of an end surface of the second log;
automatically identify growth characteristics of the second log based on the second image; and
provide instructions to categorize the second based on the growth characteristics of the second log.
